(19) United States
(12) Patent Application Publication
(10) Pub. No.: US 2014/0228402 A1
(43) Pub. Date: Aug. 14, 2014

(54) Combinations Comprising a S1P receptor modulator

(71) Applicants: Matthias Meergans, Nuernberg (DE); Ferenc Tracik, Nuernberg (DE); Katrin Schuh, Nuernberg (DE)

(72) Inventors: Matthias Meergans, Nuernberg (DE); Ferenc Tracik, Nuernberg (DE); Katrin Schuh, Nuernberg (DE)

(73) Assignee: Novartis AG, Basel (CH)

(21) Appl. No.: 14/343,104

(22) PCT Filed: Sep. 7, 2012
(86) PCT No.: PCT/EP2012/067557

§ 371 Date: Mar. 6, 2014

Related U.S. Application Data
(60) Provisional application No. 61/534,126, filed on Sep. 13, 2011.

(51) Int. Cl. A61K 31/138; A61K 45/06

(52) U.S. Cl. CPC A61K 31/138; A61K 45/06
USPC 514/321; 514/653; 514/647; 514/469; 514/651; 514/654

(57) ABSTRACT

The invention relates to a combination which comprises fingolimod and at least antidepressant compound.

The invention relates to a combination, such as a combined preparation or pharmaceutical composition, respectively, which comprises a S1P receptor modulator, such as fingolimod, and at least one anti-depressive compound, for simultaneous, separate or sequential use; use of such a combination in the prevention, delay of progression or treatment of depression, in particular in patients affected by multiple sclerosis; use of such a combination for the preparation of a pharmaceutical preparation for the prevention, delay of progression or treatment of depression, in particular in patients affected by multiple sclerosis; method of prevention, delay of progression or treatment of depression, in particular in patients affected by multiple sclerosis.

According to the invention, the anti-depressive compound may be selected from the group consisting of serotonin-norepinephrine reuptake inhibitor (SNRI), selective serotonin reuptake inhibitor (SSRI), atypical antidepressants, tricyclic antidepressants (TCAs), and monoamine oxidase inhibitors (MAOIs); in particular at least one anti-depressive compound selected from the group consisting of venlafaxine ((RS)-1-[2-dimethylamino-1-(4-methoxyphenyl)-ethyl]cyclohexanol), sertraline ((1S,4S)-4-(3,4-dichlorophenyl)-N-methyl-1,2,3,4-tetrahydronaphthalen-1-amine), escitalopram; citalopram ((RS)-1-[3-(dimethylamino)propyl]-1-(4-fluorophenyl)-1,3-dihydroisobenzofuran-5-carbonitrile), paroxetine ((3S,4R)-3-[(2H-1,3-benzodioxol-5-yloxy)methyl]-4-(4-fluorophenyl)piperidine) or fluoxetine.

Multiple Sclerosis (MS) is a chronic, demyelinating, immune-mediated disease of the central nervous system affecting 2.3 times as many women as men. With regard to the pathogenesis, MS is characterized by inflammation and destruction of myelin and axons. Typically recurrent acute episodes (relapses) of neurological symptoms, which are followed by a complete or partial recovery, can be observed during the relapsing remitting multiple sclerosis (RRMS) disease course. Approximately 50% of these patents progress to secondary progressive MS (SPMS) within 10 years, 90% within 25 years. Apart from these initially relapsing forms of MS 10-15% of patients present with primary progressive MS (PPMS) which is characterized by steady deterioration of impairment without prior experience of relapses.

Depression is one of the most important determinants of quality of life in MS. About 48% of MS patients are affected by mental comorbidity, most frequently depression (46%). Despite growing body of evidence that mental comorbidity is common in MS, it is still undertreated. Especially with regard to decreased MS therapy adherence, treatment of depression would be beneficial in order to improve adherence. Not only depression may prevent the patient from willing to be treated, but depression may also aggravate the multiple sclerosis symptoms.

There is no drug treatment for this indication in MS patients

Therefore, one therapeutic focus is on preventing or treating depression, in particular in patients affected by multiple sclerosis. Presently available agents need to be improved in order to better meet this therapeutic challenge.

Fingolimod (FTY720) Gilenya is a new chemical entity for once daily oral administration, which has received a marketing authorization for MS inter alia in USA and Europe. Fingolimod is a sphingosine 1-phosphate receptor modulator and acts in large part by down-modulating S1P/S1P receptor responses in the immune- and central nervous system.

The term “S1P receptor modulator” used herein means a compound or composition which acts ads an agonist or antagonist of a S1P receptor. According to the present invention the preferred S1P receptor modulator is FTY720 (fingolimod), i.e. 2-amino-2-[2-(4-octylphenyl) ethyl]propane-1,3-diol, in free form or in a pharmaceutically acceptable salt form, or a phosphate derivative thereof.

In an embodiment of the invention, the S1P receptor modulator is FTY720 hydrochloride, as shown below:

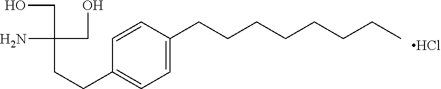 [SMILES: CCCCCCCCc1ccc(CCC(N)(CO)CO)cc1 ; Cl]

Another specific S1P receptor modulator of the invention is the phosphate derivative of fingolimod (also called FTY720-phosphate), as shown below:

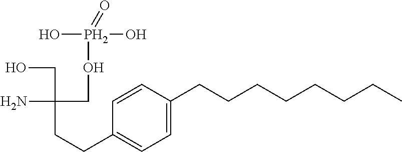 [SMILES: CCCCCCCCc1ccc(CCC(N)(CO)COP(=O)(O)O)cc1]

The present invention relates to a combination which comprises a S1P receptor agonist, such as fingolimod, in free or pharmaceutically acceptable salt form or in form of a phosphate derivative thereof, and at least one antidepressant compound or the pharmaceutically acceptable salt of such a compound, and optionally at least one pharmaceutically acceptable carrier; for simultaneous, separate or sequential use.

Preferably, the anti-depressive compound is a serotonin-norepinephrine reuptake inhibitor (SNRI), a selective serotonin reuptake inhibitor (SSRI), an atypical antidepressant, a tricyclic antidepressant (TCA), or a monoamine oxidase inhibitor (MAOI). For example the anti-depressive compound is selected from the group consisting of venlafaxine ((RS)-1-[2-dimethylamino-1-(4-methoxyphenyl)-ethyl]cyclohexanol), sertraline ((1S,4S)-4-(3,4-dichlorophenyl)-N-methyl-1,2,3,4-tetrahydronaphthalen-1-amine), escitalopram ((S)-(+)-1-[3-(dimethylamino)propyl]-1-(p-fluorophenyl)-5-phthalancarbonitrileoxalate), citalopram ((RS)-1-[3-(dimethylamino)propyl]-1-(4-fluorophenyl)-1,3-dihydroisobenzofuran), paroxetine ((3S,4R)-3-[(2H-1,3-benzodioxol-5-yloxy)methyl]-4-(4-fluorophenyl)piperidine), fluoxetine ((RS)-N-methyl-3-phenyl-3-[4-(trifluoromethyl)phenoxy]-propan-1-amine), and pharmaceutically acceptable salts thereof.

In one embodiment, the antidepressant compound is venlafaxine or a pharmaceutically acceptable salt thereof. Venlafaxine can be administered in the form as it is marketed e.g. under the trademark Effexor or Efexor.

In another embodiment, the antidepressant compound is sertraline or a pharmaceutically acceptable salt thereof, e.g. hydrochloride. Sertraline can be administered in the form as it is marketed e.g. under the trademark Zoloft or Lustral.

In a further embodiment, the antidepressant compound is escitalopram or a pharmaceutically acceptable salt thereof. Escitalopram can be administered in the form as it is marketed e.g. under the trademark Lexapro, Cipralex or Lexam.

In yet another embodiment, the antidepressant compound is citalopram or a pharmaceutically acceptable salt thereof. Citalopram ((RS)-1-[3-(dimethylamino)propyl]-1-(4-fluorophenyl)-1,3-dihydroisobenzofuran-5-carbonitrile) can be administered in the form as it is marketed e.g. under the trademark Celepram, Celexa or Cipramil.

In yet a further embodiment, the antidepressant compound is fluoxetine ((RS)-N-methyl-3-phenyl-3-[4-(trifluoromethyl)phenoxy]-propan-1-amine) or a pharmaceutically acceptable salt thereof, e.g. chlorhydrate. Fluoxetine can be administered in the form as it is marketed e.g. under the trademark Prozac, Sarafem or Fontex.

In yet a further embodiment, the antidepressant compound is paroxetine ((3S,4R)-3-[(2H-1,3-benzodioxo1-5-yloxy)methyl]-4-(4-fluorophenyl)piperidine), or a pharmaceutically acceptable salt thereof. Paroxetine can be administered in the form as it is marketed e.g. under the trademark Aropax or Paxil.

The structure of the active agents identified by code nos., generic or trade names may be taken from the actual edition of the standard compendium “The Merck Index” or from databases, e.g. Patents International (e.g. IMS World Publications). The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

The compounds to be combined can be present as pharmaceutically acceptable salts. If these compounds have, for example, at least one basic center, they can form acid addition salts. Corresponding acid addition salts can also be formed having, if desired, an additionally present basic center. The active ingredient or a pharmaceutically acceptable salt thereof may also be used in form of a hydrate or include other solvents used for crystallization.

The present invention further relates to the use of the combination as herein above defined for preventing or treating depression, depressive disorder or anxiety disorder. Depression as herein defined encompasses in particular mild and moderate depression.

The term “prevention” means prophylactic administration of the combination to healthy patients to prevent the outbreak of the conditions mentioned herein. Moreover, the term “prevention” means prophylactic administration of such combination to patients being in a pre-stage of the conditions, especially depression, e.g. patients being affected by multiple sclerosis, to be treated.

The term “delay of progression” used herein means administration of the combination, such as a combined preparation or pharmaceutical composition, to patients being in a pre-stage of the condition, especially depression, to be treated in which patients a pre-form of the corresponding condition is diagnosed or patients being affected by multiple sclerosis.

The present invention also relates to the use of the combination as herein above defined for preventing or treating depression, depressive disorder or anxiety disorder, in patients affected by MS, e.g. RRMS or PPMS, e.g. RRMS.

A combined preparation which comprises fingolimod in free form, in a pharmaceutically acceptable salt form or as a phosphate derivative, and at least one anti depressive compound, and optionally at least one, i.e., one or more, e.g. two, pharmaceutically acceptable carrier for simultaneous, separate or sequential use is especially a “kit of parts” in the sense that the components, fingolimod in free form, in a pharmaceutically acceptable salt form or as a phosphate derivative, and at least one anti depressive compound, can be dosed independently or by use of different fixed combinations with distinguished amounts of the components, i.e. at different time points or simultaneously. The parts of the kit of parts can then, e.g., be administered simultaneously or chronologically staggered, that is at different time points and with equal or different time intervals for any part of the kit of parts. Preferably, the time intervals are chosen such that the effect on the treated disease or condition in the combined use of the parts is larger than the effect which would be obtained by use of only any one of the components. Preferably, there is at least one beneficial effect, e.g. a mutual enhancing of the effect of fingolimod in free form, in a pharmaceutically acceptable salt form or as a phosphate derivative, and at least one anti depressive compound, additional advantageous effects, less side effects, a combined therapeutical effect in a non-effective dosage of one or each of the components, and especially a synergism, e.g. a more than additive effect, between fingolimod in free form, in a pharmaceutically acceptable salt form or as a phosphate derivative, and at least one anti depressive compound.

The nature of conditions mediated by fingolimod, especially multiple sclerosis, is multifactorial. Under certain circumstances, drugs with different mechanisms of action may be combined. However, just considering any combination of drugs having different mode of action but acting in the similar field does not necessarily lead to combinations with advantageous effects.

Further benefits are that lower doses of the individual drugs to be combined according to the present invention can be used to reduce the dosage, for example, that the dosages need not only often be smaller but are also applied less frequently, or can be used in order to diminish the incidence of side effects. This is in accordance with the desires and requirements of the patients to be treated.

The pharmaceutical compositions according to the invention can be prepared in a manner known per se and are those suitable for enteral, such as oral or rectal, and parenteral administration to mammals (warm-blooded animals), including humans, comprising a therapeutically effective amount of at least one pharmacologically active combination partner alone, e.g. as indicated above, or in combination with one or more pharmaceutically acceptable carriers or diluents, especially suitable for enteral or parenteral application.

Suitable pharmaceutical compositions contain, for example, from about 0.1% to about 99.9%, preferably from about 1% to about 60%, of the active ingredient(s). Pharmaceutical preparations for the combination therapy for enteral or parenteral administration are, for example, those in unit dosage forms, such as sugar-coated tablets, tablets, capsules or suppositories, or ampoules. If not indicated otherwise, these are prepared in a manner known per se, for example by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

In particular, a therapeutically effective amount of each of the combination partner of the combination of the invention may be administered simultaneously or sequentially and in any order, and the components may be administered separately or as a fixed combination. For example, the method of delay of progression or treatment of a proliferative malignant disease according to the invention may comprise (i) administration of the first agent a) in free or pharmaceutically acceptable salt form and (ii) administration of a co-agent b) in free or pharmaceutically acceptable salt form, simultaneously or sequentially in any order, in jointly therapeutically effective amounts, preferably in synergistically effective amounts, e.g. in daily or intermittently dosages corresponding to the amounts described herein. The individual combination partners of the combination of the invention may be administered separately at different times during the course of therapy or concurrently in divided or single combination forms. Furthermore, the term administering also encompasses the use of a pro-drug of a combination partner that convert in vivo to the combination partner as such. The instant invention is therefore to be understood as embracing all such regimens of simultaneous or alternating treatment and the term “administering” is to be interpreted accordingly.

The effective dosage of each of the combination partners employed in the combination of the invention may vary depending on the particular compound or pharmaceutical composition employed, the mode of administration, the condition being treated, the severity of the condition being treated. Thus, the dosage regimen of the combination of the invention is selected in accordance with a variety of factors including the route of administration and the renal and hepatic function of the patient. A physician, clinician or veterinarian of ordinary skill can readily determine and prescribe the effective amount of the single active ingredients required to prevent, counter or arrest the progress of the condition. Optimal precision in achieving concentration of the active ingredients within the range that yields efficacy without toxicity requires a regimen based on the kinetics of the active ingredients' availability to target sites.

Daily dosages for fingolimod will, of course, vary depending on a variety of factors, for example the compound chosen, the particular condition to be treated and the desired effect. In general, however, satisfactory results are achieved on administration of fingolimod at daily dosage rates of the order of ca. 0.1 to 100 mg as a single dose or in divided doses, e.g. 0.5 mg daily. Fingolimod may be administered by any conventional route, in particular enterally, e.g. orally, e.g. in the form of tablets, capsules, drink solutions or parenterally, e.g. in the form of injectable solutions or suspensions. Suitable unit dosage forms for oral administration comprise from ca. 0.1 to 30 mg component (a), e.g. 0.1 to 25 mg, together with one or more pharmaceutically acceptable diluents or carriers therefor.

Venlafaxine may be administered orally to a human in a dosage range varying from about 75 to 225 mg/day. Sertraline may be administered orally to a human in a dosage range varying from about 25 to 200 mg/day. Escitalopram may be administered orally to a human in a dosage range varying from about 10 to 20 mg/day. Citalopram may be administered orally to a human in a dosage range varying from about 20 to 60 mg/day. Paroxetine may be administered orally to a human in a dosage range varying from about 20 to 60 mg/day. Fluoxetine may be administered orally to a human in a dosage range varying from about 10 to 60 mg/day, e.g. 20 to 50 mg/day.

It can be shown by established test models and especially those test models described herein that the combination of fingolimod in free form, in a pharmaceutically acceptable salt form or as a phosphate derivative, and at least one anti-depressant compound results in a more effective prevention or preferably treatment of depression, in particular in patients affected by multiple sclerosis.

The person skilled in the pertinent art is fully enabled to select a relevant animal test model to prove the hereinbefore and hereinafter indicated therapeutic indications and beneficial effects. The pharmacological activity may, for example, be demonstrated in a clinical study as described hereinafter.

### Clinical Double-Blind, Randomized, Parallel-Group Study in Subjects With Relapse Remitting Multiple Sclerosis (RRMS) and Mild to Moderate Depression

Fingolimod 0.5 mg per capsule is used to be taken p.o. once daily. Following a minimum 2 weeks fingolimod treatment patients (approximately 500 patients) receive an antidepressant venlafaxine, citalopram, fluoxetine or sertralin for 16 weeks. For all antidepressant a titration period of at least 7 days, maximal 14 days, is required in which the starting doses are increased to their final doses, i.e. 150 mg venlafaxine, 40 mg citaloram, 40 mg fluoxetine, 100 mg sertralin. The antidepressant is taken p.o once daily. Patients start with the minimum dose, as follow: 75 mg venlafaxine, 20 mg citalopram, 20 mg fluoxetin, 50 mg sertralin. Dosage is then increased to their individual final dose given once daily.

The patient takes one capsule of the study medication (1× daily fingolimod and lx antidepressant drug, preferably at the same time every day, with or without food.

The study population consists of a representative group of RRMS patients with a clinical diagnosis of depression according to ICD-10 criteria and symptoms of a mild-moderate depression are assessed by Beck Depression Inventory Second Edition (BDI-II) (score between 14 and 28, inclusive).

The patients are patients with RRMS defined by 2010 revised McDonald criteria.

The therapeutic effect on depression is assessed by using the Hamilton rating Scale for Depression (HRSD) (also known as Hamilton Depression Rating Scale), that is a multiple choice questionnaire used by physicians to rate the severity of a patient's major depression. The questionnaire rates the severity of symptoms observed in depression such as low mood, insomnia, agitation, anxiety and weight loss.

BDI-II is a 21-item self-report instrument intended to assess the existence and severity of symptoms of depression as listed in the American Psychiatric Association's Diagnostic and Statistical Manual of Mental Disorders Fourth Edition (DSM-IV, 1994). Items indicate increases and decreases in sleep and appetite items label agitations, concentration difficulty and loss of energy. Each of the 21 item corresponding to a symptom of depression is summed to give a single score for the BDI-II. There is a four-point scale for each item ranging from 0 to 3. Total score of 0-13 is considered minimal range, 14-19 is mild, 20-28 is moderate and 29-63 is severe.

Preferably, the jointly therapeutically effective amounts of fingolimod in free or pharmaceutically acceptable salt form or as a phosphate derivative thereof, and at least one further pharmaceutically active compound are administered simultaneously or sequentially in any order, separately or in a fixed combination.

It is one objective of this invention to provide a pharmaceutical composition comprising a quantity, which is jointly therapeutically effective against depression, in particular in MS patients, of fingolimod (i) in free form, a pharmaceutically acceptable salt form or as form of a phosphate derivative and (ii) at least one anti-depressant compound and at least one pharmaceutically acceptable carrier.

The pharmaceutical compositions according to the invention can be prepared in a manner known per se and are those suitable for enteral, such as oral or rectal, and parenteral administration to mammals (warm-blooded animals), including man, comprising a therapeutically effective amount of the pharmacologically active compound, alone or in combination with one or more pharmaceutically acceptable carries, especially suitable for enteral or parenteral application.

The novel pharmaceutical preparations contain, for example, from about 10% to about 100%, e.g., 80% or 90%, preferably from about 20% to about 60%, of the active ingredient. Pharmaceutical preparations according to the invention for enteral or parenteral administration are, for example, those in unit dose forms, such as sugar-coated tablets, tablets, capsules or suppositories, and furthermore ampoules. These are prepared in a manner known per se, for example by means of conventional mixing, granulating, sugar-coating, dissolving or lyophilizing processes. Thus, pharmaceutical preparations for oral use can be obtained by combining the active ingredient with solid carriers, if desired granulating a mixture obtained, and processing the mixture or granules, if desired or necessary, after addition of suitable excipients to give tablets or sugar-coated tablet cores.

In this composition, components (i) and (ii) can be administered together, one after the other or separately in one combined unit dose form or in two separate unit dose forms. In one preferred embodiment of the invention, the unit dose form is a fixed combination. In a fixed combination the components (i) and (ii) are administered in the form of a single galenic formulation, e.g. a single tablet, capsule or a single infusion.

A further aspect of the present invention is the use of a pharmaceutical composition comprising fingolimod, in free form, pharmaceutically acceptable salt or as a phosphate derivative, and at least one anti-depressant, in each case in free form or in form of a pharmaceutically acceptable salt thereof for the preparation of a pharmaceutical preparation for the prevention or treatment of depression, in particular in MS patients, e.g. RRMS patients.

A therapeutically effective amount of each of the components of the combination of the present invention may be administered simultaneously or sequentially and in any order, and the components may be administered separately or as a fixed combination. For example, the method of treatment of the invention may comprise (i) administration of fingolimod, in free form, pharmaceutically acceptable salt or as a phosphate derivative, and (ii) administration of at least one anti-depressant compound simultaneously or sequentially in any order, in jointly therapeutically effective amounts, preferably in synergistically effective amounts, e.g. in daily dosages corresponding to the ratios described herein.

The invention relates in particular to a commercial package comprising jointly therapeutically effective amounts of fingolimod, in free form, a pharmaceutically acceptable salt thereof or a phosphate derivative thereof, and at least one anti-depressant compound together with instructions for use thereof in the treatment of depression, in particular MS patients.

A further aspect of the present invention is a method of treating depression, in particular in MS patients, comprising administering to a warm-blooded animal in need thereof jointly therapeutically effective amounts of fingolimod, in free form, a pharmaceutically acceptable salt thereof or a phosphate derivative thereof, and at least one anti-depressant compound. Preferably, in this method of treating the active ingredients are administered simultaneously or sequentially in any order, separately or in a fixed combination.

Furthermore, the present invention provides a method of treating depression, in particular in MS patients, comprising administering to a warm-blooded animal in need thereof jointly therapeutically effective amounts of fingolimod, in free form, a pharmaceutically acceptable salt thereof or a phosphate derivative thereof, and at least one anti-depressant compound.

The weight ratio of the daily doses of fingolimod or a pharmaceutically acceptable salt thereof or a phosphate derivative thereof, to at least one anti-depressant compound may vary within wide limits depending in particular on the needs of the warm-blooded animal treated.

Furthermore there is provided a method to improve adherence to MS therapy comprising administering at least one anti-depressant compound to the patients affected by multiple sclerosis, e.g. one anti-depressant compound as hereinabove defined.

The present invention also provides a method for increasing, improving, or maintaining multiple sclerosis treatment compliance in a population of patients affected by multiple sclerosis comprising administering to said population a solid pharmaceutical composition suitable for oral administration, comprising administering at least one anti-depressant compound to the patients, e.g. one anti-depressant compound as hereinabove defined. Furthermore, there is provided a method for increasing, improving or maintaining multiple sclerosis treatment adherence in a population of multiple sclerosis patients, or preventing such patients from quitting or stopping multiple sclerosis treatment, comprising administering to said population a solid pharmaceutical composition suitable for oral administration, comprising administering at least one anti-depressant compound to the patients, e.g. one anti-depressant compound as hereinabove defined.

The following Example shall illustrate the invention described above; they are not, however, intended to limit the scope of the invention in any way.

### EXAMPLE

### Soft Capsules

| | | |
|---|---|---|
| Fingolimod, HCl | 30 | mg |
| Polyethylene glycol 300 | 300 | mg |
| Polysorbate 80 | 20 | mg |
| Total | 350 | mg |

All references, including U.S., World and EP Patents and applications referred to herein are hereby incorporated by reference in their entirety as if set forth in full herein.

1. Combination which comprises fingolimod in free form, in a pharmaceutically acceptable salt form or as a phosphate derivative, and at least one antidepressant compound or a pharmaceutically acceptable salt thereof and optionally at least one pharmaceutically acceptable carrier; for simultaneous, separate or sequential use.

2. Combination according to claim 1 wherein the antidepressant compound is selected from the group consisting of serotonin-norepinephrine reuptake inhibitor (SNRI) and serotonin reuptake inhibitor (SSRI).

3. Combination according to claim 1 wherein the antidepressant compound is selected from the group consisting of venlafaxine, sertraline, escitalopram, citalopram, fluoxetine, paroxetine, and the pharmaceutically acceptable salts thereof.

4. Combination according to claim 1 which is a combined preparation or a pharmaceutical composition.

5. Combination according to claim 4 which is a combined preparation for simultaneous, separate or sequential use in the prevention, delay of progression or treatment of depression.

6. Combination according to claim 4 which is a combined preparation for simultaneous, separate or sequential use in the prevention, delay of progression or treatment of depression in patients affected by multiple sclerosis.

7. Combination according to claim 1 comprising fingolimod hydrochloride.

8. Method of treating depression, in particular in patients affected by multiple sclerosis, comprising administering to a patient in need thereof jointly therapeutically effective amounts of i) fingolimod, in free form, pharmaceutically acceptable salt or phosphate derivative form, and ii) at least one anti-depressive compound in free or pharmaceutically acceptable salt, preferably selected from the group consisting of serotonin-norepinephrine reuptake inhibitor (SNRI) and serotonin reuptake inhibitor (SSRI), or the pharmaceutically acceptable salts of such compounds.

9. A pharmaceutical composition comprising a quantity which is jointly therapeutically effective against depression of a combination according to any one of claims 1, and at least one pharmaceutically acceptable carrier.

10. A method for the prevention, delay of progression or treatment of depression, in particular in patients affected by multiple sclerosis, comprising administering the combination according to claim 1.

11. A commercial package comprising as active agents a combination according to any one of claims 1 together with instructions for simultaneous, separate or sequential use thereof in the prevention, delay of progression or treatment of depression, in particular in patients affected by multiple sclerosis.
